(19) United States
(12) Patent Application Publication
(10) Pub. No.: US 2010/0180899 A1
(43) Pub. Date: Jul. 22, 2010

(54) AIR FLOW REGULATING DEVICE

(76) Inventor: Michael Capezzuto, Fort Pierce, FL (US)

Correspondence Address: L.C. BEGIN & ASSOCIATES, PLLC, 510 HIGHLAND AVENUE, PMB 403, MILFORD, MI, 48381, US

(21) Appl. No.: 12/610,553

(22) Filed: Nov. 2, 2009

Related U.S. Application Data
(60) Provisional application No. 61/146,021, filed on Jan. 21, 2009.

(51) Int. Cl. A61M 16/20

(52) U.S. Cl. 128/205.24; 128/207.16

(57) ABSTRACT

One embodiment of an air flow regulating device may include a first chamber, a first one-way valve, a second chamber, and a second one-way valve. The first chamber may include an intermediate member having a tapered opening, a first hollow adaptor may be coupled to a ventilation tube for receiving air, and a second hollow adaptor may be coupled to an endotracheal tube for delivering the air to a patient. The first one-way valve may be carried within an end portion of the intermediate member. The second chamber may enclose the first chamber for configuring a hollow space therebetween. The second one-way valve may be configured between the first chamber and the second chamber for regulating a flow of the air through the tapered opening toward the hollow space, and to vibrate the air upon emerging from tapered opening.

### CROSS REFERENCE TO RELATED APPLICATION

This application claims the benefit of U.S. Provisional Application No. 61/146,021 filed on Jan. 21, 2009, the disclosure of which is incorporated by reference.

### FIELD OF THE DISCLOSURE

The present disclosure generally relates to breathing apparatuses, and, more particularly to an air flow regulating device to be used in conjunction with an endotracheal tube for regulating a flow of the air therethrough.

### BACKGROUND OF THE DISCLOSURE

Use of breathing apparatuses, such as ventilators and ambu bags, may be common in medical institutions. Generally, a ventilator may be use to facilitate an individual who is physically unable to breathe, or breathing insufficiently. Typically, a ventilator may include a ventilation tube which may carry air and an endotracheal tube which may be inserted into patient's trachea and connected to the ventilation tube for receiving the air, to be delivered to the patient's lungs.

During the utilization of the ventilator, it has been observed that mucus plugs or internal secretions may get caught in the endotracheal tube. Due to such clogging, a flow of air may get blocked, which may lead to insufficient breathing of the patient. Further, the patients, breathing with the help of the ventilator, are unable to cough or dislodge the mucus plugs and the internal secretions from their lungs. Therefore, the medical attendants, such as nurses, may have to clean the endotracheal tubes for maintaining the flow of the air through the endotracheal tubes. Otherwise, the medical attendants may have to do the suction of the mucus plugs and the internal secretions from the patient's lungs. The task of cleaning the endotracheal tube and suction of the mucus plugs and the internal secretions may be very inconvenient and time consuming. Moreover, in case, when the mucus plugs and the internal secretions remain stagnant in the patient's lungs, the patient may suffer from an infection. For example, bacteria in the lungs may begin to multiply, causing the patient to suffer from ventilator associated pneumonia, which may also deteriorate the health of the patient.

### SUMMARY OF THE DISCLOSURE

One embodiment of an air flow regulating device may include a first chamber. The first chamber may include an intermediate member having a tapered opening. The first chamber may also include a first hollow adaptor extending from a first end portion of the intermediate member and adapted to be coupled to a ventilation tube for receiving air from a ventilator. The first chamber may also include a second hollow adaptor extending from a second end portion opposite to the first end portion of the intermediate member. The second hollow adaptor may include a perforated portion having a plurality of holes, and an unperforated portion extending from the perforated portion and adapted to be coupled to an endotracheal tube. The air flow regulating device may also include a first one-way valve carried within the second end portion of the intermediate member. The first one-way valve may be adapted to restrict a flow of the air toward the endotracheal tube through the second hollow adaptor. Further, the first one-way valve may be adapted to allow a flow of exhaled air carried by the endotracheal tube from a patient toward the first chamber through the second hollow adaptor.

The air flow regulating device may also include a second chamber adapted to sealingly enclose the first chamber for configuring a hollow space therebetween. The second chamber may be adapted to enclose the intermediate member and the perforated portion of the second hollow adaptor. The air flow regulating device may also include a second one-way valve configured between the first chamber and the second chamber for regulating the flow of the air between the ventilation tube and the endotracheal tube. The air may be received by the intermediate member of the first chamber through the first hollow adaptor and may be allowed to pass through the tapered opening toward the hollow space. The second one-way valve may be adapted to regulate the flow of the air toward the hollow space and adapted to vibrate the air emerging from the tapered opening. The vibrating air may be received by the endotracheal tube through the plurality of holes for being delivered to the patient.

Another embodiment of an air flow regulating device may include a first chamber. The first chamber may include an intermediate member having a tapered opening. The first chamber may also include a first hollow adaptor extending from a first end portion of the intermediate member and adapted to be coupled to a ventilation tube for receiving air from a ventilator. The first chamber may also include a second hollow adaptor extending from a second end portion opposite to the first end portion of the intermediate member. The second hollow adaptor may include a perforated portion having a plurality of holes and an unperforated portion extending from the perforated portion and adapted to be coupled to an endotracheal tube. The air flow regulating device may also include a sealing member carried within the second end portion of the intermediate member. The sealing member may be adapted to restrict a flow of the air toward the endotracheal tube through the second hollow adaptor.

The air flow regulating device may also include a second chamber adapted to sealingly enclose the first chamber for configuring a hollow space therebetween. The second chamber may be adapted to enclose the intermediate member and the perforated portion of the second hollow adaptor. The air flow regulating device may also include a one-way valve configured between the first chamber and the second chamber for regulating the flow of the air between the ventilation tube and the endotracheal tube. The air may be received by the intermediate member of the first chamber through the first hollow adaptor and may be allowed to pass through the tapered opening toward the hollow space. The one-way valve may be adapted to regulate the flow of the air toward the hollow space and may be adapted to vibrate the air emerging from the tapered opening. The vibrating air may be received by the endotracheal tube through the plurality of holes for being delivered to the patient.

### BRIEF DESCRIPTION OF THE DRAWINGS

The advantages and features of the present disclosure will become better understood with reference to the following detailed description and claims taken in conjunction with the accompanying drawing, in which:

FIG. 1 is an unassembled perspective view of one embodiment of an air flow regulating device;

FIG. 2 is an assembled perspective view of the air flow regulating device of FIG. 1 mounted between a ventilation tube and an endotracheal tube;

FIG. 3 is a cross-sectional view the air flow regulating device of FIG. 2 along an axis 3-3′;

FIG. 4 is a cross-sectional view of another embodiment of an air flow regulating device mounted between the ventilation tube and the endotracheal tube;

FIG. 5 is an unassembled perspective view of yet another embodiment of an air flow regulating device;

FIG. 6 is a cross-sectional view of the air flow regulating device of FIG. 5 mounted between the ventilation tube and the endotracheal tube; and

FIG. 7 is a cross-sectional view of still another embodiment of an air flow regulating device mounted between the ventilation tube and the endotracheal tube.

Like reference numerals refer to like parts throughout the description of several views of the drawings.

### DETAILED DESCRIPTION OF THE DISCLOSURE

The exemplary embodiments described herein detail for illustrative purposes are subject to many variations in structure and design. It should be emphasized, however, that the present disclosure is not limited to a particular air flow regulating device as shown and described. It is understood that various omissions and substitutions of equivalents are contemplated as circumstances may suggest or render expedient, but these are intended to cover the application or implementation without departing from the spirit or scope of the claims of the present disclosure. Also, it is to be understood that the phraseology and terminology used herein is for the purpose of description and should not be regarded as limiting.

The terms, “first,” “second,” and the like, herein do not denote any order, elevation or importance, but rather are used to distinguish one element with another. Further, the terms, “a” and “an” herein do not denote a limitation of quantity, but rather denote the presence of at least one of the referenced item.

The present disclosure may provide an air flow regulating device. The air flow regulating device may be configured in between a ventilation tube carrying an air from a ventilator, and an endotracheal tube which may be inserted into a patient's trachea for delivering the air into patient's lungs. The air flow regulating device may enable in regulating a flow of the air to be delivered to the patient. Further, the air flow regulating device may enable in vibrating the air to be received by the patient.

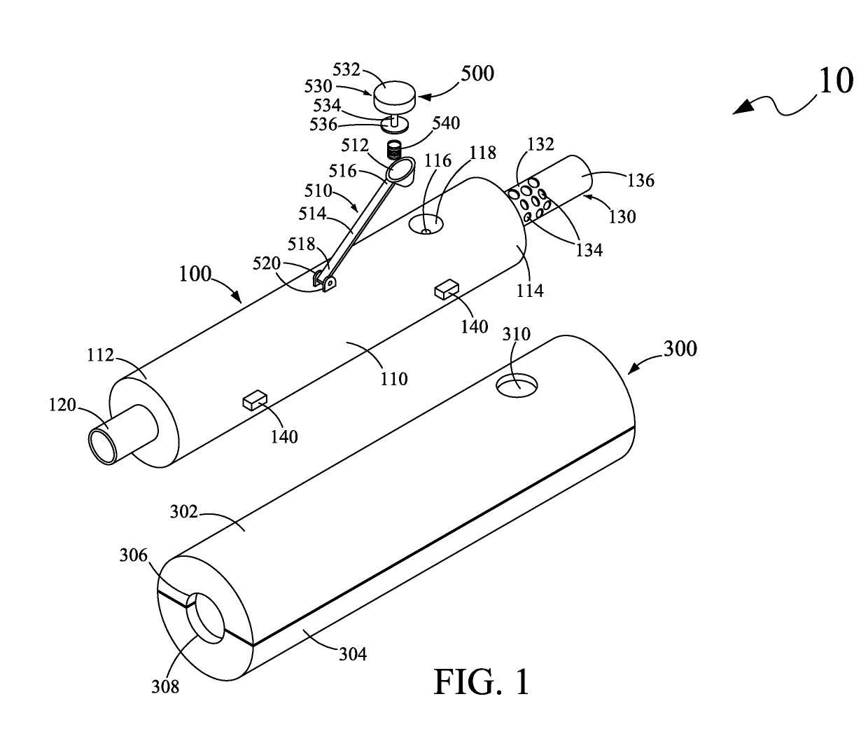

Referring to FIGS. 1-3, one embodiment of an air flow regulating device 10 (“device”) may include a first chamber 100. The first chamber 100 may include an intermediate member 110 that in one form may be a hollow elongated cylinder. Of course, the intermediate member 110 may have other suitable shapes, such as a hollow elongated oval shape or a hollow elongated polygonal shape.

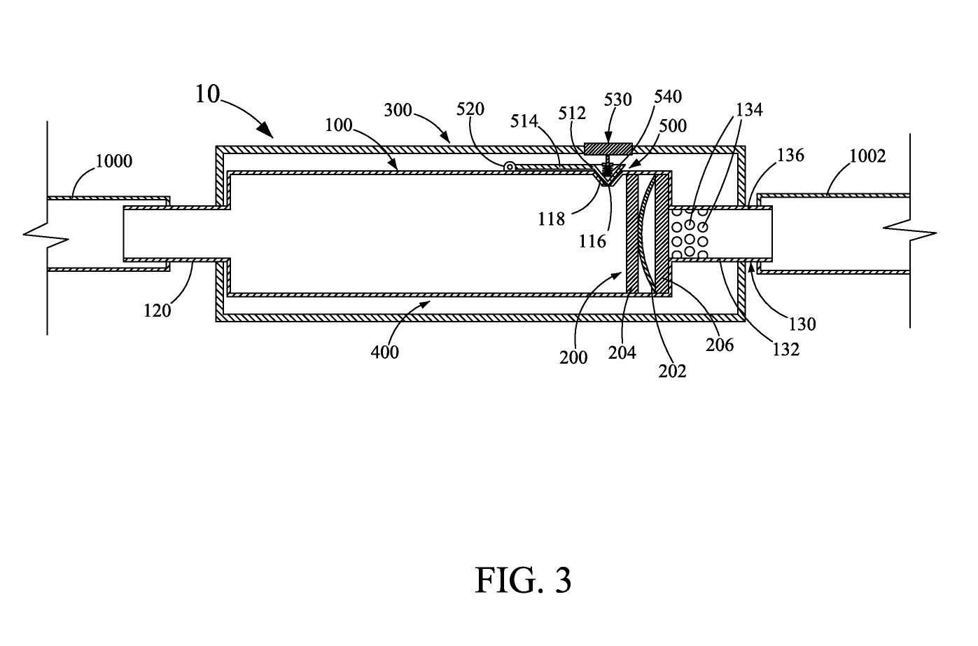

The intermediate member 110 may include a first end portion 112 and a second end portion 114 opposite to the first end portion 112. The intermediate member 110 may also include a tapered opening 116 configured, in proximity to the second end portion 114. The opening 116 may be defined by a frustum shaped structure 118 extending inwardly within the intermediate member 110, best shown in FIG. 3. Further, the frustum shaped structure 118 may be a funnel shaped structure, without funnel's stem. Of course, the opening 116 may not be tapered but rather have edges or walls that are position parallel or unparallel to provide various other shapes.

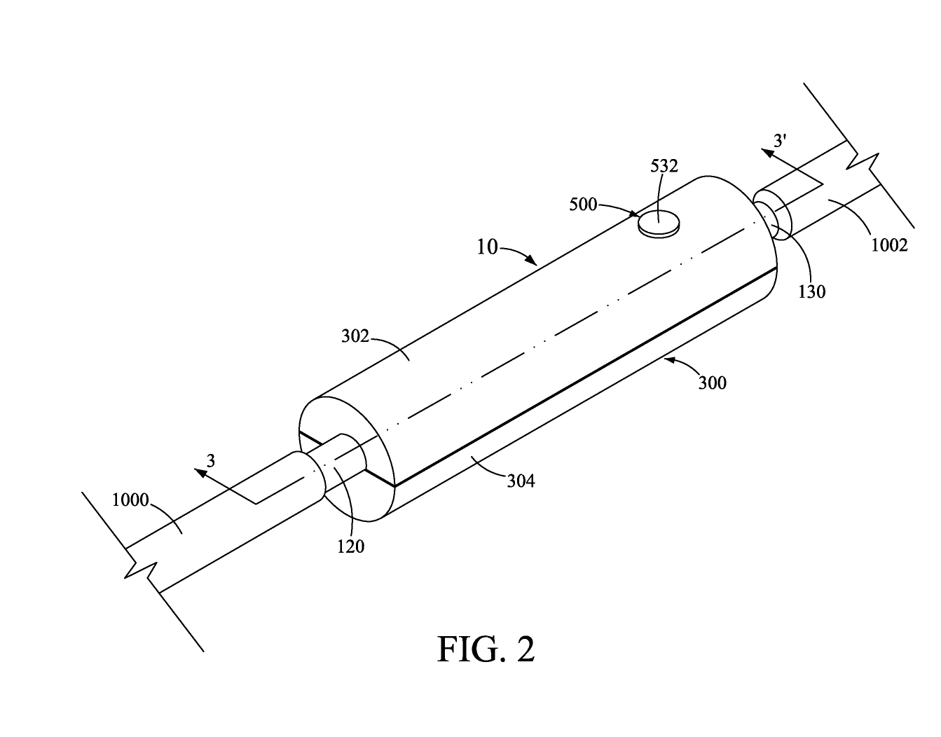

The first chamber 100 may also include a first hollow adaptor 120 that may extend from the first end portion 112 of the intermediate member 110. The first hollow adaptor 120 in this form may be a hollow elongated cylinder. Of course, the adaptor 120 may have other suitable shapes. The first hollow adaptor 120 may be coupled to a ventilation tube 1000, as shown in FIGS. 2 and 3. Accordingly, the first hollow adaptor 120 may enable in receiving an air from a ventilator (not shown) into the intermediate member 110 of the first chamber 100 through the ventilation tube 1000.

The first chamber 100 may also include a second hollow adaptor 130 that may extend from the second end portion 114 of the intermediate member 110. The second hollow adaptor 130 may be a hollow elongated cylinder. The second hollow adaptor 130 may include a perforated portion 132 that may have a plurality of holes 134. The second hollow adaptor 130 may also include an unperforated portion 136 extending from the perforated portion 132. The unperforated portion 136 may be adapted to connect to an endotracheal tube 1002, as shown in FIGS. 2 and 3. The second hollow adaptor 130 may enable in delivering the air to a patient through the endotracheal tube 1002 which may be inserted into patient's trachea.

As best shown in FIG. 3, the device 10 may also have a first one-way valve 200 that may be carried within the second end portion 114 of the intermediate member 110. The first one-way valve 200 may restrict the flow of air from the intermediate member 110 through the second hollow adaptor 130 and toward the endotracheal tube 1002. However, the first one-way valve 200 may direct a flow of exhaled gas carried by the endotracheal tube 1002 from the patient toward the first chamber 100.

Specifically, the first one-way valve 200 may be configured to only allow unidirectional flow of the exhaled gas from the endotracheal tube 1002, through the second hollow adaptor 130 and into the intermediate member 110. The first one-way valve 200 in one form may include one or more movable rubber flaps 202 that passively spread apart from each other in response to flow in one direction and then sealingly close together in response to flow in the opposite direction. Such a valve may function in a similar manner as a heart valve. However, the flaps may be made of any suitable material. Further, the one-way valve may be a spring-loaded ball valve or various other suitable check valves.

As best shown in FIG. 3, the flaps 202 may extend inwardly toward the first hollow adaptor 120. Accordingly, the flaps 202 may be sealingly engaged with each other to prevent or limit the flow of air from the first chamber 100 through the second hollow adaptor 130 and toward the endotracheal tube 1002. In addition, the flaps 202 may spread apart from each other to permit exhaled gas to flow from the endotracheal tube 1002 through the second hollow adaptor 130, and into the first chamber 100. As shown in FIG. 3, the first one-way valve 200 may be supported between support members 204,206 in the second end portion 114 of the intermediate member 110. Of course, the first one-way valve 200 may be supported in the second end portion 114 by other suitable means, such as adhesive.

As shown in FIGS. 1 and 3, the device 10 may also include a second chamber 300. The second chamber 300 in one form may be a hollow elongated cylinder, similar to the intermediate member 110 of the first chamber 100. Of course, the second chamber 300 may have other shapes, which may be similar to the shape of the intermediate member 110 of the first chamber 100. In addition, the second chamber 300 may have a first half portion 302 and a second half portion 304 pivotally coupled to each other. Each of the first and second half portions 302, 304, may have a larger diameter as compared to the intermediate member 110 of the first chamber 100. Therefore, the second chamber 300 may sealingly enclose the first chamber 100 for configuring a hollow space 400 therebetween and also enclosing the perforated portion 132 of the second hollow adaptor 130 therein.

Referring back to FIG. 1, the first and second half portions 302, 304 of the second chamber 300 may include semicircular cutouts for accommodating a respective one of the first and second hollow adaptors 120,130 therebetween. For example, the first and second half portions 302, 304 may include semicircular cutouts 306 and 308, receptively, which may be capable of accommodating the first hollow adaptor 120 therebetween, as shown in FIG. 2. Similarly, the first and second half portions 302, 304 may also include semicircular cutouts (not shown) which may be capable of accommodating the second hollow adaptor 130 therebetween.

Further, the second chamber 300 may be carried by the first chamber 100. Specifically, the first chamber 100 may include connectors 140 carried by the intermediate member 110. The connectors 140 may enable in retaining the second chamber 300 in a position which sealingly encloses the intermediate member 110 and the perforated portion 132 within the second chamber 300. The connectors 140 may be an attachment known in the art, such a mating flange/groove with a seal.

As shown in FIG. 1, the device 10 may also include a second one-way valve 500 that may be associated between the first chamber 100 and the second chamber 300 for regulating the flow of the air between the ventilation tube 1000 and the endotracheal tube 1002.

The second one-way valve 500 may include a cap 510 operatively mounted on the intermediate member 110 of the first chamber 100. The cap 510 may be adapted to close the opening 116. Specifically, the cap 510 may include a plug 512 adapted to be received within the opening 116 and sealingly close the opening 116. The plug 512 in this form may have a conical shape, such that the plug 512 may be received within the frustum shaped structure 118. Moreover, the plug 512 may be made of rubber to facilitate in sealing the opening 116. Of course, the plug may be made of other suitable materials.

The second one-way valve 500 may also include an arm 514 carrying the plug 512. The arm 514 may be pivotally mounted on the intermediate member 110 of the first chamber 100. Specifically, a first end portion 516 of the arm 514 may be carrying the plug 512 and a second end portion 518 opposite to the first end portion 516 may be pivotally mounted on the intermediate member 110. Further, the second end portion 518 of the arm 514 may be pivotally mounted on the intermediate member 110 with the help of coupling members 520, which in this form may be pins, spindles or other protrusions pivotally attached to the intermediate member.

The second one-way valve 500 may also include a regulating knob 530 operatively mounted on the second chamber 300. The regulating knob 530 may include a head portion 532 adapted to be carried by the second chamber 300. More specifically, the head portion 532 may be operatively carried by an opening 310 configured on the first half portion 302 of the second chamber 300. Further, the head portion 532 may be threadably carried by the opening 310 of the second chamber 300. Accordingly, the head portion 532 may be rotated such that the regulating knob 530 rotates about the opening 310. The regulating knob 530 may also include a connecting member 534 that may be carried by the head portion 532 and a base portion 536 carried by the connecting member 534.

The second one-way valve 500 may also include a spring member 540 disposed in between the plug 512 and the base portion 536. The spring member 540 may restrict a movement of the plug 512, when the air tends to pass through the opening 116 by moving away the plug 512 from the opening 116. Therefore, the plug 512 may vibrate when the air emerges from the opening 116 and received in the hollow space 400.

Further, the head portion 532 of the regulating knob 530 may be adapted to be rotated for adjusting a distance between the plug 512 and the base portion 536, and thereby adjusting tension of the spring member 540 received therebetween. By adjusting the tension of the spring member 540 a frequency of the vibrating movement of the cap 510 may be regulated. Moreover, by adjusting the distance between the plug 512 and the base portion 536 the flow of vibrating air through the opening 116 may be also regulated.

For example, when the head portion 532 may be rotated for reducing the distance between the plug 512 and the base portion 536, the tension of the spring member 540 may be increased for decreasing a frequency of the vibration of the cap 510. Further, the decreased vibration frequency of the cap 510 may cause the air to less vibrate. Moreover, by reducing the distance between the plug 512 and the base portion 536, a reduced amount of low vibrating air may be allowed to pass through the opening 116.

Alternatively, when the head portion 532 may be rotated for increasing the distance between the plug 512 and the base portion 536, the tension of the spring member 540 may be reduced for increasing the frequency of the vibration of the cap 510. Therefore, the increased vibration frequency of the cap 510 may cause the air to more vibrate. Further, by increasing the distance between the plug 512 and the base portion 536, an increased amount of high vibrating air may be allowed to pass through the opening 116.

Referring now to FIGS. 2 and 3, in use, the device 10 may be in communication between the ventilation tube 100 and the endotracheal tube 1002. The air may flow from the ventilation tube 1000 through the first hollow adaptor 120 and into the intermediate member 110 of the first chamber 100. The first one-way valve 200 may block the flow of air from the intermediate member 110 through the second hollow adaptor 130 and toward the endotracheal tube 1002.

Further, the second one-way valve 500, particularly the plug 512 of the cap 510, may emerge from the opening 116 in response to the pressure of the air, thereby imparting vibration to the air as the second one-way valve 500 may permit the air to flow into the hollow space 400. Thereafter, the vibrating air may flow through the plurality of holes 134 of the second hollow adaptor 130 and into the endotracheal tube 1002 for being delivery to the patient. The vibrating air may eliminate or reduce mucus plugs that may block the endotracheal tube 1002. Accordingly, the device 10 may provide a uniform flow of the air and reduce the possibility of infections.

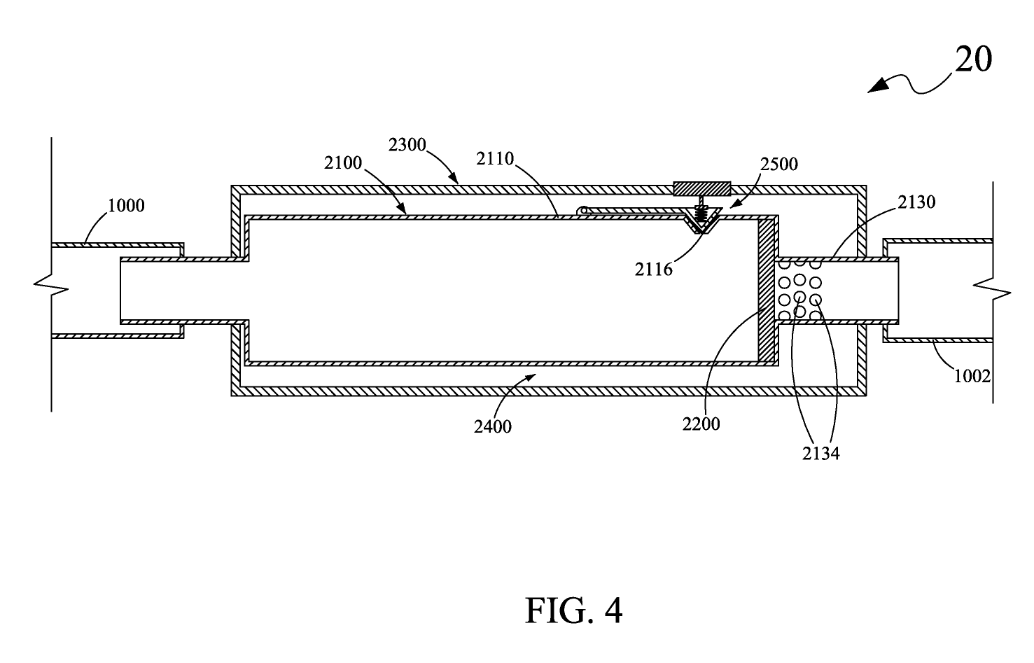

Referring now to FIG. 4, another embodiment of an air flow regulating device 20 (“device”) may include a first chamber 2100, a second chamber 2300, and a one-way valve 2500 similar to the first chamber 100, the second chamber 300, and the second one-way valve 500 of the device 10. However, the device 20 may include a sealing member 2200 instead of the first one-way valve 200 of the device 10.

The sealing member 2200 may be carried within the first chamber 2100. More specifically, the sealing member 2200 may be carried within an end portion of an intermediate member 2110 of the first chamber 2100. For example, the sealing member 2200 may be carried by the intermediate member 2110 in a similar manner as the first one-way valve 200 may be carried by the intermediate member 110.

The sealing member 2200 may be adapted to restrict a flow of air toward a second hollow adaptor 2130. For example, the sealing member 2200 may restrict the flow of the air in a similar manner as the first one-way valve 200 does. Accordingly, the air may be allowed to pass through a tapered opening 2116 toward a hollow space 2400. Further, the one-way valve 2500 may regulate the flow of air, received from the ventilation tube 1000, toward the hollow space 2400 and may vibrate the air upon emerging from the tapered opening 2116. Therefore, the vibrating air may be received by the endotracheal tube 1002 through a plurality of holes 2134 configured on the second hollow adaptor 2130 of the first chamber 2100 for being delivered to a patient.

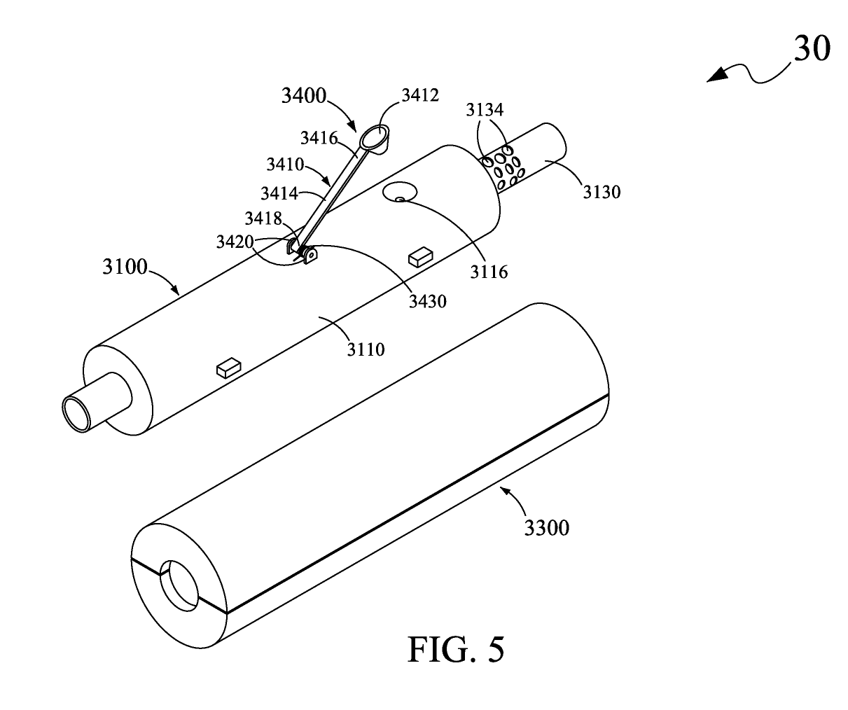

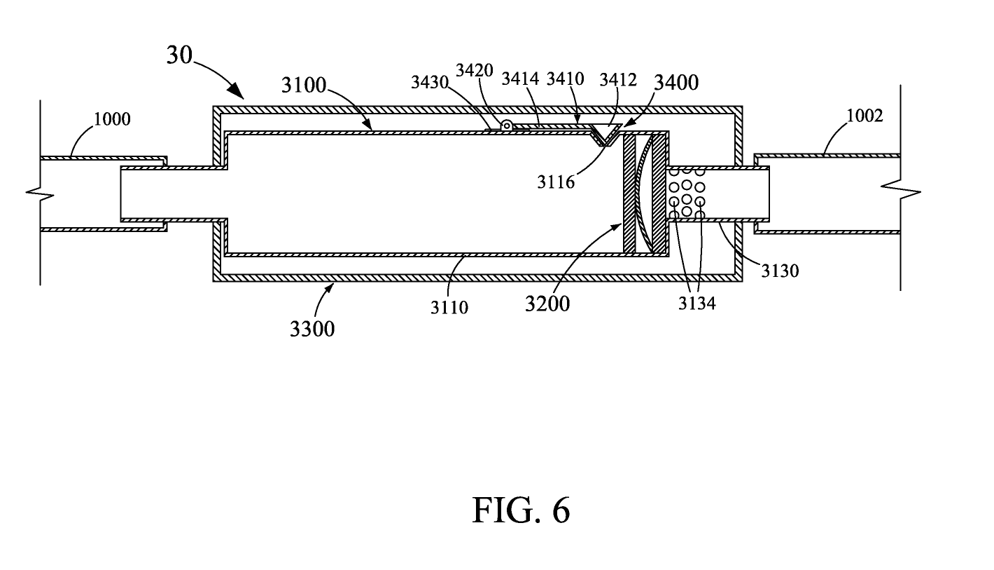

Referring now to FIGS. 5 and 6, yet another embodiment of an air flow regulating device 30 (“device”) may include a first chamber 3100, a first one-way valve 3200, and a second chamber 3300 similar to the first chamber 100, the first one-way valve 200, and the second chamber 300 of the device 10. However, the second chamber 3300 of the device 30 may lack the opening 310 configured on the first half portion 302 of the second chamber 300.

Further, the device 30 may also include a second one-way valve 3400 which may include a cap 3410 operatively mounted on an intermediate member 3110 of the first chamber 3100. The cap 3410 may include a plug 3412 which may be adapted to close a tapered opening 3116. The plug 3412 may have a conical shape and may be made of rubber for sealing the opening 3116. The cap 3410 may also include an arm 3414 carrying the plug 3412 at a first end portion 3416 thereof. Further, the arm 3414 may be pivotally mounted on the intermediate member 3110 at a second end portion 3418 opposite to the first end portion 3416. For example, the second end portion 3418 may be pivotally mounted on the intermediate member 3110 with the help of coupling members 3420, which in this form may be pins, spindles or other protrusions pivotally attached to the intermediate member.

The second one-way valve 3400 may also include a torsional spring 3430 associated to the second end portion 3418 of the arm 3414, as shown in FIG. 5. The torsional spring 3430 may restrict a movement of the cap 3410 when air, received through the ventilation tube 1000, tend to pass through the opening 3116 and thereby causing the cap 3410 to vibrate. Accordingly, vibrating air may be received by the endotracheal tube 1002 through a plurality of holes 3134 configured on a second hollow adaptor 3130 of the first chamber 3100 for being delivered to a patient. The device 30 of the present embodiment may provide a smoother outer surface of the second chamber 3300 because the second one-way valve 3400 lacks the knob 530, which is operatively mounted on the second chamber 300 of the device 10.

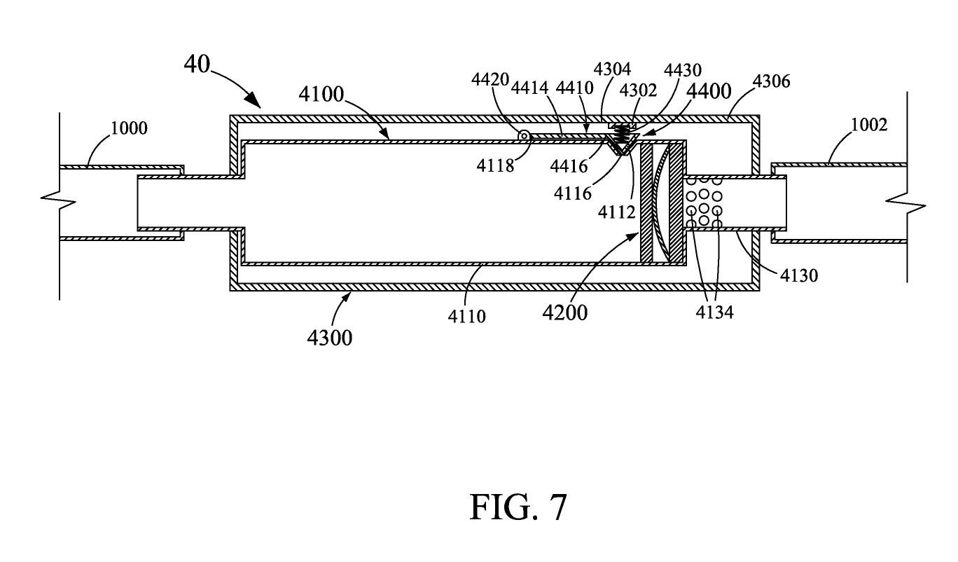

Referring now to FIG. 7, still another embodiment of an air flow regulating device 40 (“device”) may include a first chamber 4100 and a first one-way valve 4200 similar to the first chamber 100 and the first one-way valve 200 of the device 10. The device 40 may also include a second chamber 4300 similar to the second chamber 300 of the device 10. However, the second chamber 4300 may lack the opening 310 configured on the first half portion 302 of the second chamber 300. Further, the second chamber 4300 may include a guide member 4302 which may extend radially inward from an inner surface 4304 of the second chamber 4300. Specifically, the guide member 4302 may extend from a first half portion 4306 of the second chamber 4300.

The device 40 may further include a second one-way valve 4400 which may include a cap 4410 operatively mounted on an intermediate member 4110. The cap 4410 may include a plug 4412 which may be adapted to close a tapered opening 4116. The plug 4412 may have a conical shape and may be made of rubber for sealing the opening 4116. The cap 4410 may also include an arm 4414 carrying the plug 4412 at a first end portion 4416 thereof. The arm 4414 may be pivotally mounted on the intermediate member 4110 at a second end portion 4418 opposite to the first end portion 4416. For example, the second end portion 4418 may be pivotally mounted on the intermediate member 4110 with the help of coupling members 4420.

The second one-way valve 4400 may also include a helical spring 4430 carried between the guide member 4302 and the plug 4412. The helical spring 4430 may tend to restrict a movement of the cap 4410 when air, received trough the ventilation tube 1000, may tend to pass through the opening 4116 and thereby causing the cap 4410 to vibrate. The vibrating air may be received by the endotracheal tube 1002 through a plurality of holes 4134 configured on a second hollow adaptor 4130 of the first chamber 4100 for being delivered to a patient.

Based on the foregoing description of the present disclosure, an air flow regulating device, such as the devices 10-40, may be capable of delivering a vibrating air to a patient. Further, the device may be capable of being adjusted for regulating a flow of the vibrating air and thereby providing a required flow of vibrating air to the patient. The vibrating air may enable in avoiding/reducing mucus plugs or internal secretions to get caught in an endotracheal tube. Accordingly, the device of the present disclosure may enable in preventing a patient from an infection, such as ventilator associated pneumonia, which may occur due to the stagnation of the mucus plug and the secretion in the patient's lungs. Further, the device of the present disclosure may enable in avoiding/reducing an inconvenient and time consuming task of cleaning the endotracheal tubes, which needs to be performed by the medical attendants.

The foregoing descriptions of specific embodiments of the present disclosure have been presented for purposes of illustration and description. They are not intended to be exhaustive or to limit the present disclosure to the precise forms disclosed, and obviously many modifications and variations are possible in light of the above teaching. The embodiments were chosen and described in order to best explain the principles of the present disclosure and its practical application, to thereby enable others skilled in the art to best utilize the present disclosure and various embodiments with various modifications as are suited to the particular use contemplated. It is understood that various omission and substitutions of equivalents are contemplated as circumstance may suggest or render expedient, but such are intended to cover the application or implementation without departing from the spirit or scope of the claims of the present disclosure.

What is claimed is:

1. An air flow regulating device comprising:
a first chamber comprising,
an intermediate member having a tapered opening,
a first hollow adaptor extending from a first end portion of the intermediate member and adapted to be coupled to a ventilation tube for receiving air from a ventilator, and
a second hollow adaptor extending from a second end portion opposite to the first end portion of the intermediate member, the second hollow adaptor comprising a perforated portion having a plurality of holes and an unperforated portion extending from the perforated portion and adapted to be coupled to an endotracheal tube;
a first one-way valve carried within the second end portion of the intermediate member, the first one-way valve adapted to restrict a flow of the air toward the endotracheal tube through the second hollow adaptor, the first one-way valve adapted to allow a flow of exhaled air carried by the endotracheal tube from a patient toward the first chamber through the second hollow adaptor;
a second chamber adapted to sealingly enclose the first chamber for configuring a hollow space therebetween, the second chamber adapted to enclose the intermediate member and the perforated portion of the second hollow adaptor; and
a second one-way valve configured between the first chamber and the second chamber for regulating the flow of the air between the ventilation tube and the endotracheal tube,
wherein the air is received by the intermediate member of the first chamber through the first hollow adaptor and is allowed to pass through the tapered opening toward the hollow space, and
wherein the second one-way valve is adapted to regulate the flow of the air toward the hollow space and adapted to vibrate the air emerging from the tapered opening, and
wherein the vibrating air is received by the endotracheal tube through the plurality of holes for being delivered to the patient.

2. The air flow regulating device of claim 1, wherein the tapered opening is defined by a frustum shaped structure.

3. The air flow regulating device of claim 1, wherein the second one-way valve comprises
a cap operatively mounted on the intermediate member, the cap adapted to close the tapered opening;
a regulating knob operatively mounted on the second chamber; and
a spring member disposed between the cap and the regulating knob, the spring member tend to restrict a movement of the cap when the air passes through the tapered opening and thereby causing the cap to vibrate,
wherein the regulating knob is adapted to be rotated for adjusting a distance between the cap and the regulating knob thereby adjusting tension of the spring member, and
wherein by adjusting the tension of the spring member the vibrating movement of the cap is adjusted, and the flow of vibrating air through the tapered opening is regulated.

4. The air flow regulating device of claim 3, wherein the cap comprises
a plug adapted to close the tapered opening; and
an arm carrying the plug at a first end portion thereof, the arm pivotally mounted on the intermediate member at a second end portion opposite to the first end portion.

5. The air flow regulating device of claim 4, wherein the plug is having a conical shape.

6. The air flow regulating device of claim 4, wherein the plug is made of rubber.

7. The air flow regulating device of claim 1, wherein the second one-way valve comprises
a cap operatively mounted on the intermediate member, the cap comprising
a plug adapted to close the tapered opening, and
an arm carrying the plug at a first end portion thereof, the arm pivotally mounted on the intermediate member at a second end portion opposite to the first end portion; and
a torsional spring associated to the second end portion of the arm, the torsional spring tend to restrict a movement of the cap when the air passes through the tapered opening and thereby causing the cap to vibrate.

8. The air flow regulating device of claim 7, wherein the plug is having a conical shape.

9. The air flow regulating device of claim 7, wherein the plug is made of rubber.

10. The air flow regulating device of claim 1, wherein the second chamber comprises a guide member extending radially inward from an inner surface thereof.

11. The air flow regulating device of claim 10, wherein the second one-way valve comprises
a cap operatively mounted on the intermediate member, the cap comprising
a plug adapted to close the tapered opening, and
an arm carrying the plug at a first end portion thereof, the arm pivotally mounted on the intermediate member at a second end portion opposite to the first end portion; and
a helical spring carried between the guide member and the plug, the helical spring tend to restrict a movement of the cap when the air passes through the tapered opening and thereby causing the cap to vibrate.

12. The air flow regulating device of claim 11, wherein the plug is having a conical shape.

13. The air flow regulating device of claim 11, wherein the plug is made of rubber.

14. An air flow regulating device comprising:
a first chamber comprising,
an intermediate member having a tapered opening,
a first hollow adaptor extending from a first end portion of the intermediate member and adapted to be coupled to a ventilation tube for receiving air from a ventilator, and
a second hollow adaptor extending from a second end portion opposite to the first end portion of the intermediate member, the second hollow adaptor comprising a perforated portion having a plurality of holes and an unperforated portion extending from the perforated portion and adapted to be coupled to an endotracheal tube;
a sealing member carried within the second end portion of the intermediate member, the sealing member adapted to restrict a flow of the air toward the endotracheal tube through the second hollow adaptor;
a second chamber adapted to sealingly enclose the first chamber for configuring a hollow space therebetween, the second chamber adapted to enclose the intermediate member and the perforated portion of the second hollow adaptor; and
an one-way valve configured between the first chamber and the second chamber for regulating the flow of the air between the ventilation tube and the endotracheal tube,
wherein the air is received by the intermediate member of the first chamber through the first hollow adaptor and is allowed to pass through the tapered opening toward the hollow space, and
wherein the one-way valve is adapted to regulate the flow of the air toward the hollow space and adapted to vibrate the air emerging from the tapered opening, and
wherein the vibrating air is received by the endotracheal tube through the plurality of holes for being delivered to the patient.

15. The air flow regulating device of claim 14, wherein the tapered opening is defined by a frustum shaped structure.

16. The air flow regulating device of claim 14, wherein the one-way valve comprises
a cap operatively mounted on the intermediate member, the cap adapted to close the tapered opening;
a regulating knob operatively mounted on the second chamber; and
a spring member disposed between the cap and the regulating knob, the spring member tend to restrict a movement of the cap when the air passes through the tapered opening and thereby causing the cap to vibrate,
wherein the regulating knob is adapted to be rotated for adjusting a distance between the cap and the regulating knob thereby adjusting tension of the spring member, and
wherein by adjusting the tension of the spring member the vibrating movement of the cap is adjusted, and the flow of vibrating air through the tapered opening is regulated.

17. The air flow regulating device of claim 16, wherein the cap comprises
a plug adapted to close the tapered opening; and
an arm carrying the plug at a first end portion thereof, the arm pivotally mounted on the intermediate member at a second end portion opposite to the first end portion.

18. The air flow regulating device of claim 17, wherein the plug is having a conical shape.

19. The air flow regulating device of claim 17, wherein the plug is made of rubber.
